(19) United States
(12) Patent Application Publication
(10) Pub. No.: US 2013/0111632 A1
(43) Pub. Date: May 2, 2013

(54) OVERPRODUCTION OF JASMONIC ACID IN TRANSGENIC PLANTS

(71) Applicants: Antony Champion, Quissac (FR); Michel Nicole, Teyran (FR); Patrick Doumas, Montpellier (FR)

(72) Inventors: Antony Champion, Quissac (FR); Michel Nicole, Teyran (FR); Patrick Doumas, Montpellier (FR)

(73) Assignee: INSTITUT NATIONAL DE LA RECHERCHE AGRONOMIQUE, Paris (FR)

(21) Appl. No.: 13/703,897

(22) PCT Filed: Jun. 16, 2011
(86) PCT No.: PCT/EP11/60026

§ 371 Date: Dec. 12, 2012

(30) Foreign Application Priority Data
Jun. 16, 2010 (FR) 10 54754

(51) Int. Cl. C12N 15/82

(52) U.S. Cl. CPC C12N 15/8281; C12N 15/8243
USPC 800/279; 800/298; 800/301; 800/302; 800/317; 800/320; 800/314; 800/317.3; 800/320.2; 800/317.4; 435/419; 800/278; 800/294

(57) ABSTRACT

The present invention relates to methods for obtaining a transgenic plant that overproduces jasmonic acid, and optionally OPDA, a jasmonic acid precursor. Said methods include transforming the plant using a nucleic acid encoding ORA47. The accumulation of jasmonic acid confers in particular to the transformed plant an improved resistance to pathogenic agents. The transgenic plants obtained may also be used for the production of pharmaceutically important secondary metabolites whose synthesis is induced by jasmonates.

### RELATED APPLICATIONS

The present application claims priority to French Patent Application number FR 10 54 754 filed on Jun. 16, 2010. The content of the French Patent Application is incorporated herein by reference in its entirety.

### BACKGROUND OF THE INVENTION

The fight against plant diseases is one of the main concerns in agriculture. Worldwide, it is estimated that about one third of harvests is destroyed, either in the field or during storage, by pathogenic agents (insects, viruses, bacteria, oomycetes, or fungi). This results in significant economic losses and, in certain regions of the world, may lead to population under-feeding or malnutrition.

Over time, several approaches have been developed to fight plant diseases, the oldest being a chemical approach using pesticides, such as fungicides, bactericides, nematicides, viricides and insecticides. Although efficient, the use of molecules from the chemical industry is generally associated with pollution problems and potential risks for animal and/or human health. Furthermore, several phytopathogens of viral or bacterial origin are not sensitive to chemical products that are currently available on the market, and some pathogenic fungi, acari, nematodes and insects can bear high doses of pesticides without harmful effects. Different alternatives to chemical treatments have since been developed and are now commonly used in the field or are in the process of being introduced on the market. It is worth citing in particular: the biological approach which employs natural predator organisms against pathogenic agents of plants; the genetic improvement of plants which is still one of the methods of choice when resistance or tolerance genes have been identified and can be introduced in the genetic material of plants; and the methods that make use of the plants natural resistance to diseases caused by bioagressors

The natural resistance of plants to diseases caused by bioagressors is often initiated by the specific recognition of a given pathogen, and particularly by the recognition of molecules called “elicitors” or “effectors” that are present on the surface of pathogens or that are excreted by said pathogens. This recognition leads to the rapid induction of the plant's defense mechanisms which limit the multiplication and propagation of the pathogen in different plant tissues. Some studies have shown that the application of elicitors on a plant increases the plant's resistance to bioagressors via preventive activation of the plant's defense reactions. Stimulation of plants natural defense has opened up a novel avenue for the development of new approaches in the fight against plant diseases and is attracting more and more interest. However, the use of elicitors presents the disadvantage of having a limited action spectrum and a cost that is still too high.

The recognition of elicitors generally results in the biosynthesis of one or more secondary molecules of cell signalling, the most important of these secondary molecules being: jasmonic acid (Memelink et al., Trends Plant Sci., 2001, 6: 212-219; Turner et al., Plant Cell, 2002, 14 (suppl.): S153-S164), ethylene (Wang et al., Plant Cell, 2002, 14 (suppl.): S131-S151; Guo et al., Curr. Opin. Plant Biol., 2004, 7: 40-49) and salicylic acid (Shah, Curr. Opin. Plant Biol., 2003, 6: 365-371). The production of these secondary molecules generates a signalling pathway leading to a cascade of events that are responsible for the physiologic adaptation of plants to external stress. Current studies aim at identifying signals that activate all or some of the elements of natural defense with the goal of developing new phytosanitary strategies that are more respectful of the environment than chemical methods.

Some research studies have focussed on jasmonic acid which plays an important role in the interactions between plants and insects, plants and bacteria, and plants and fungi by increasing the plant defense (McConn et al., Proc. Natl. Acad. Sci. USA, 1997, 94: 5473-5477; Dong, Curr. Opin. Plant Biol., 1998, 1: 316-323; Penninckx et al., Plant Cell, 1998, 10: 2103-2113; Thomma et al., Proc. Natl. Acad. Sci. USA, 1998, 95: 15107-15111; Vijayan et al., Proc. Natl. Acad. Sci. USA, 1998, 95: 7209-7214). Jasmonic acid and its precursors and derivatives, collectively called “jasmonates”, constitute a family of compounds that are synthesized from membrane linoleic acid via the octadecanoids metabolic pathway (Turner et al., Plant Cell, 2002, 14 (suppl.): S153-S164; Atallah et al., In Encyclopedia of Plant and Crop Science, 2004, R. M. Goodman (Ed), New York: Marcel Dekker Inc, pp. 1006-1009). The development, in Arabidopsis, of mutants that exhibit deficiencies in some jasmonate-dependent mechanisms, has revealed the complexity of the signalling pathway of jasmonic acid. This pathway is marked out by its autoregulation—indeed, the genes of the jasmonic acid synthetic pathway may themselves be activated by jasmonates (Bonaventure et al., Plant J., 2007, 49: 889-898). More than ten genes, called ORA genes, encoding transcription factors of the AP2/ERF domain of Arabidopsis and whose expression is jasmonate-dependent have been identified (Atallah, PhD thesis, “Jasmonate-responsive AP2-domain transcription factors in Arabidopsis”, 2005, Netherlands). The role of some of these genes (in particular ORA59, ORA47 and ORA37) in the signalling pathway of jasmonic acid has since been elucidated (Pre, PhD thesis, “ORA EST: Functional analysis of jasmonate-responsive AP2/ERF-domain transcription factors in Arabidopsis thaliana”, 2006, Netherlands).

Progresses made in this field have allowed for a better understanding of the role of jasmonic acid in the plant natural resistance. However, the molecular bases of the regulation mechanisms that control the signalling pathway of jasmonic acid are still unclear and too poorly understood for a logical and informed development of new strategies for fighting against plant diseases to be possible.

### SUMMARY OF THE INVENTION

The present invention generally relates to the improvement of plant and in particular to the improvement of plant natural defense against bioagressors, via an increase in the production of jasmonic acid in these plants.

In Arabidopsis thaliana, it is known that the overproduction of the transcription factor, ORA47, does not lead to accumulation of jasmonic acid in the plant but only to an accumulation of a jasmonic acid precursor called OPDA (12-oxo-phytodienoic acid) (see Chapter 3 of Dr. Pré's thesis). The inventors have shown that in the cotton plant, overexpression of this transcription factor surprisingly induced a strong accumulation of not only OPDA but also of jasmonic acid. Indeed, in a cotton plant transformed with the gene ORA47, the level of accumulation of OPDA and of jasmonic acid were found to be multiplied by a factor of 50 and by a factor of 140, respectively when compared to a control transformation (i.e. transformation with an empty vector). By comparison in Arabidopsis thaliana, the level of accumulation of OPDA was found to be multiplied by a factor of 2 compared to a control transformation while no accumulation of jasmonic acid was detected. These results suggest that a few locks that control the synthesis of jasmonic acid in Arabidopsis via ORA47 expression are unlocked in a heterologous context.

Consequently, given the important role of jasmonic acid in the mechanisms of plant defense against bioagressors, the present inventors propose that the heterologous overexpression of ORA47 may be used to increase the level of jasmonic acid in plants and to thereby generate plants that exhibit an improved resistance to pathogenic agents. This approach has several advantages compared to existing methods: (1) it is universal, (2) it induces an accumulation of jasmonic acid within the plant, and (3) it exploits natural defense mechanisms of plants with the aim of increasing resistance and therefore is a strategy that is more respectful of the environment than the chemical methods used for fighting plant diseases.

Therefore, in a first aspect, the present invention relates to a transgenic plant comprising an exogenous nucleic acid sequence that allows expression of ORA47, wherein the expression of ORA47 induces, within the plant, an overproduction or accumulation of jasmonic acid. Preferably, the nucleic acid sequence that allows expression of ORA47 is integrated within the genome of the transgenic plant. In certain embodiments, the overproduction of jasmonic acid is accompanied with an overproduction or accumulation of OPDA, a jasmonic acid precursor. Preferably, the plant that is transformed does not belong to the Arabidopsis thaliana species.

In certain embodiments, a transgenic plant according to the invention exhibits an improved resistance to at least one pathogenic agent compared to a plant of the same species that has not been transformed according to the present invention. The at least one pathogenic agent may be selected from the group consisting of bacteria, viruses, fungi, insects and oomycetes that are able to induce a disease in a plant, and any combination thereof.

In certain embodiments, the transgenic plant belongs to the Malvaceae family (e.g., cotton, cocoa, okra, etc. . . . ), to the Solanaceae family (e.g., tobacco, tomato, potato, eggplant, etc. . . . ), to the Rubiaceae family (e.g., coffee, etc. . . . ), to the Poaceae or Gramineae family (e.g., rice, corn, wheat, barley, oat, rye, mil, sugarcane, etc. . . . ) or to the Vitaceae family (e.g., vine, etc. . . . ). In certain preferred embodiments, the transgenic plant belongs to the Gossypium or Cotoneaster genuses (cotton), to the Nicotiona genus (tobacco), to the Oryza genus (rice), to the Solanum genus (tomato), to the Coffea genus (coffee), or to the Vitis genus (vine). In certain preferred embodiments, the transgenic plant belongs to the Gossypium or Cotoneaster genuses (cotton). In other preferred embodiments, the transgenic plant belongs to the Nicotiona genus (tobacco).

In another aspect, the present invention relates to a vegetal material obtained or extracted from a transgenic plant according to the invention. The vegetal material may be a plant cell, a culture of plant cells, a protoplast, a plant organ, a plant callus, a plant seed, a plant leave, a plant stem, a plant root, a flower, a fruit, a tuber, pollen, or a plant cutting. In certain preferred embodiments, the vegetal material can be used to regenerate a whole plant.

In another aspect, the present invention relates to methods for obtaining a transgenic plant according to the invention.

In certain embodiments, the method comprises steps of: (a) transforming a plant cell with an expression construct or expression vector comprising a nucleic acid sequence encoding ORA47 in order to obtain a plant cell stably transformed; and (b) culturing the plant cell stably transformed obtained in order to regenerate a whole plant comprising, integrated within its genome, a nucleic acid sequence allowing expression of ORA47. The step of culturing the plant cell stably transformed may comprise steps of: culturing several plant cells stably transformed in order to regenerate several whole plants, and selecting, among the regenerated whole plants, those plants that comprise, integrated within their genome, a nucleic acid sequence allowing the expression of ORA47 in plants.

In other embodiments, the method comprises steps of: (a) transforming an Agrobacterium host cell in order to obtain a recombinant host cell; and (b) transforming a plant or a plant cell via infection with the recombinant host cell obtained in order to obtain a whole plant comprising, integrated within its genome, a nucleic acid sequence allowing the expression of ORA47. Step (b) may comprise: infecting several plants or several plant cells with recombinant host cells, optionally culturing several infected plant cells in order to regenerate several whole plants; and selecting, among the infected plants or among the regenerated plants, those plants that comprise, integrated within their genome, a nucleic acid sequence allowing expression of ORA47 in plants.

In yet another aspect, the invention relates to the use of an expression construct or expression vector comprising a nucleic acid sequence allowing expression or synthesis of ORA47 in a plant, to induce in said plant, an overproduction or accumulation of jasmonic acid, and optionally an overproduction or accumulation of OPDA.

In certain embodiments, the plant overproducing jasmonic acid exhibits an improved resistance to at least one pathogenic agent compared to a plant of the same species that does not overproduce jasmonic acid. The at least one pathogenic agent may be selected from the group consisting of bacteria, viruses, fungi, insects and oömycetes that are capable of inducing a disease in a plant and any combination thereof.

It is known in the art that jasmonic acid induces, in different species of plants, the expression of genes that are involved in the biosynthesis of secondary metabolites, many of which belonging to the families of taxoids, phenylpropanoids, flavonoids, anthocynanins, guaianolides, anthroquinones, sesquiterpenoids and alkaloids, and which present an economical interest, for example as pharmaceutical compounds, as food dyes or flavors.

Consequently, in still another aspect, the present invention relates to transgenic plants according to the invention that produce at least one secondary metabolite whose biosynthesis is induced by jasmonic acid. The invention also relates to the use of methods described herein for the production of such secondary metabolites.

These and other objects, advantages and features of the present invention will become apparent to those of ordinary skill in the art having read the following detailed description of the preferred embodiments.

### BRIEF DESCRIPTION OF THE DRAWING

FIG. 1 is a picture of a leave of a cotton plant, half of which that has been transformed according to the present invention (see Example 1 for experimental details). Histochemical analysis shows the presence of indigo crystals all over the region that has been infiltrated with the bacterial strain LBA1119 p35S:Gus.

FIG. 2 is a set of two graphs showing the quantity of OPDA and of jasmonic acid measured by HPLC-MS in extracts of cotton cotyledons that have been transformed to overexpress ORA47 or not (see Example 2 for experimental details).

FIG. 3 is a set of four graphs showing the expression of four genes (GhAOS, GhOAC2, GhAOC5, and GhACX1a, which are involved in the jasmonate biosynthesis pathway) in cotton cotyledons that have been transformed to overexpress ORA47 or not (see Example 3 for experimental details).

FIG. 4 is a set of three graphs showing the expression of three genes (Ghlox1, GhERF-1Xa1 and GhERF-IXa2, which are associated to the cotton plant defense and which are inducible by exogenous jasmonate treatments) in cotton cotyledons that have been transformed to overexpress ORA47 or not (see Example 4 for experimental details).

FIG. 5 is a graph showing the Xcm20 population growth in GFP-transformed cotton and in ORA47-transformed cotton measured 1 day, 6 days and 12 days post inoculation with the bacterium Xanthomonas campestris pv. malvacearum race 20 (Xcm20).

FIG. 6 is a graph showing the quantity of jasmonic acid (JA) and of OPDA accumulated in tobacco leaves transformed with GFP (control) or with ORA47 measured 0, 24 hours and 48 hours after transformation.

FIG. 7 is a set of two graphs showing the expression (A) of the gene NbAOX and (B) of the gene NbODX in tobacco transformed with GFP (control) or with ORA47 measured 0, 24 hours and 48 hours after transformation. The genes NbAOX and NbODX encode enzymes involved in the biosynthesis pathway of nicotine.

### DETAILED DESCRIPTION OF CERTAIN PREFERRED EMBODIMENTS

As mentioned above, the present invention relates to the transformation of a plant, in order to induce, in said plant, an overproduction or accumulation of jasmonic acid. The transformation includes using an expression construct which allows, in the transformed plant, the expression or synthesis of ORA47, an AP2/ERF-type transcription expression factor of Arabidopsis thaliana. As indicated above, in the context of the present invention, the transformation is based on the heterologous overexpression of ORA47, or in other words, the transformation of the present invention is carried out in plants that do not belong to the Arabidopsis thaliana species.

### I—Expression Constructs and Vectors Allowing for the Overproduction or Accumulation of Jasmonic Acid in a Plant

An expression construct (or expression vector) according to the present invention comprises a nucleic acid sequence encoding ORA47. As mentioned above, ORA47 is a transcription factor (i.e. a protein that modulates the expression of genes) that belongs to the AP2/ERF family of Arabidopsis thaliana, and whose expression leads to the coordinated induction of several genes encoding enzymes of the biosynthesis pathway of jasmonates (Atallah, PhD thesis, 2005).

### Nucleic Acid Sequence Encoding ORA47

The gene ORA47 encoding ORA47, also called At1g74930, (GenBank Accession Number: NM_{—}106151—SEQ ID NO. 1), and the protein ORA47 (GenBank Accession Number: NP_{—}177631—SEQ ID NO. 2) have previously been isolated and sequenced.

In the practice of the present invention, the nucleic acid sequence encoding ORA47 may be any nucleic acid sequence whose transcription results in protein ORA47 (SEQ ID NO. 2) or a homologous polypeptide thereof. Preferably, the nucleic acid sequence encoding ORA47 comprises, or consists of, the sequence set forth in SEQ ID NO: 1 or a homologous sequence thereof resulting from the genetic code degeneracy. Alternatively, the nucleic acid sequence encoding ORA47 comprises, or consists of, a sequence, which is homologous to the sequence set forth in SEQ ID NO: 1 and which encodes a polypeptide homologous to ORA47. Alternatively still, the nucleic acid sequence encoding ORA47 comprises, or consists of, a nucleic acid sequence that is complementary to the sequence set forth in SEQ ID NO: 1 or a homologous sequence thereof, a nucleic acid sequence that is modified compared to the sequence set forth in SEQ ID NO: 1 or a homologous sequence thereof, or a representative fragment of any one of the preceding sequences (for example, an open reading frame).

The terms “nucleic acid sequence”, “nucleic acid”, nucleic acid molecule”, “polunucleotide” and “oligonucleotide” are used herein interchangeably. They refer to a given sequence of nucleotides, modified or not, which defines a region of a nucleic acid molecule and which may be either under the form a single strain or double strain DNAs or under the form of transcription products thereof.

The term “nucleic acid sequence homologous to the sequence set forth in SEQ ID NO: 1” refers to any nucleic acid sequence that differs from SEQ ID NO: 1 by substitution, deletion and/or insertion of one nucleotide or of a limited number of nucleotides (e.g. 2, 3, 4, 5, 6, 7, 8, 9, or 10), at positions such that the homologous nucleic acid sequence encodes a polypeptide that is homologous to ORA47, and in particular to the amino acid sequence set forth in SEQ ID NO: 2. Preferably such a homologous nucleic acid sequence has a percentage of identity such that it is identical to at least 75% of the sequence set forth in SEQ ID NO: 1, preferably at least 85%, more preferably at least 95% or more.

The term “homologous” (or “homology”), as used herein, is synonymous with the term “identity” and refers to the sequence similarity between two polypeptide molecules or between two nucleic acid molecules. When a position in both compared sequences is occupied by the same base or same amino acid residue, the respective molecules are then homologous at that position. The percentage of homology between two sequences corresponds to the number of matching or homologous positions shared by the two sequences divided by the number of positions compared and multiplied by 100. Generally, a comparison is made when the two sequences are aligned to give maximum homology. This percentage is purely statistical and the differences between the two compared sequences are spread at random and over the whole length of the sequence. The terms “optimal alignment” and “best alignment”, which are used herein interchangeably, refer to the alignment for which the percentage of identity is determined as described herein to be the highest. The optimal alignment of sequences, that is necessary to the comparison, may be performed manually or using softwares (GAP, BESTFIT, BLASTP, BLASTN, FASTA, and TFASTA, which are available either on the NCBI website, or in the Wisconsin Genetics Software Package, Genetics Computer Group, Madison, Wis.). Homologous amino acid sequences share identical or similar amino acid sequences. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g. that have a similar size, shape, electric charge, chemical properties, including the ability to form covalent or hydrogen bonds, or the like. Particularly preferred conservative substitutions are those fulfilling the criteria defined for an “accepted point mutation” as described by Dayhoff et al. (“Atlas of Protein Sequence and Structure”, 1978, Nat. Biomed. Res. Foundation, Washington, D.C., Suppl. 3, 22: 354-352).

Preferably, in the context of the present invention, a nucleic acid sequence homologous to the nucleic acid sequence set forth in SEQ ID NO: 1 specifically hybridizes to a sequence that is complementary to the sequence set forth in SEQ ID NO: 1 under stringent conditions (Sambrook et al., “Molecular Cloning—A Laboratory Manual”, Cold Spring Harbor Laboratory Press, 1989).

As used herein, the term “modified nucleic acid sequence” refers to any nucleic acid sequence that is obtained by mutagenesis using techniques well known in the art, and that comprises modifications compared to normal sequences, for example, mutations in regulatory and/or promoter sequences of the polypeptide expression, in particular leading to the modification of the expression or activity level of said polypeptide. In addition, the term “modified nucleic acid sequence” also encompasses any nucleic acid sequence encoding a modified ORA47 polypeptide.

The term “representative fragment” of a nucleic acid sequence refers to any fragment of the sequence set forth in SEQ ID NO: 1 (or of a homologous sequence thereof or of a modified sequence thereof) that encodes a polypeptide exhibiting an activity that is identical or similar to the activity of ORA47 (e.g. a fragment of ORA47). In certain embodiments, a representative fragment of SEQ ID NO: 1 is an open reading frame of said sequence (see Examples section below). One skilled in the art knows how to identify an open reading frame within a given sequence.

Techniques for isolating or cloning a gene or a nucleic acid sequence encoding a transcription factor (such as ORA47) are known in the art and include, but are not limited to, isolation from genomic DNA, preparation from complementary DNA, and combination of these methods. Cloning of a gene, or of a nucleic acid sequence encoding a transcription factor, from genomic DNA may be carried out for example using PCR (polymerase chain reaction) or via screening of expression libraries to detect cloned DNA fragments with identical structural characteristics (Innis et al., “PCR: A Guide to Method and Application”, 1990, Academic Press: New York). Other methods of amplification of nucleic acids known to those skilled in the art may be used in the context of the present invention, such as for example, ligase chain reaction (LCR), ligation activated transcription (LAT), and Nucleic Acid Sequence Based Amplification (NASBA).

### Expression Constructs

In an expression construct according to the present invention, the nucleic acid sequence encoding ORA47 is inserted in the sense orientation and is preferably linked to one or more elements that allows for its expression and optionally for its regulation in a plant or plant cell. Thus, preferably, an expression construct according to the present invention comprises 5′ and 3′ regulatory sequences operably linked to a nucleic acid sequence encoding ORA47. The term “operably linked” refers to a functional link between the 5′ and 3′ regulatory sequences and the nucleic acid sequence that they control. An expression construct according the present invention comprises, in the 5′→3′ direction of transcription, a transcription initiation sequence, the nucleic acid sequence encoding ORA47 and a transcription termination sequence, that are functional in a plant or plant cell. Such a combination is designated herein interchangeably as “nucleic acid sequence allowing expression of ORA47 in a plant” or “nucleic acid sequence allowing synthesis of ORA47 in a plant”.

Promoters.

The transcription initiation sequence is also called promoter. As used herein, the term “promoter” refers to any polynucleotide capable of regulating the expression, in a cell, of a nucleic acid sequence to which the promoter is operably linked. In the context of the invention, a promoter is capable of exerting its regulating action in a plant cell (i.e., it is a “plant promoter”). Thus, in the context of the present invention, a promoter type regulatory sequence is a regulatory region that is recognized by a RNA polymerase in a cell and that is able to initiate transcription of the nucleic acid sequence of ORA47 in a plant or plant cell. The promoter may be homologous to a cell of the host plant or, alternatively, may be heterologous to a cell of the host plant. Furthermore, the promoter may be a natural sequence (i.e., a sequence that exists in nature) or a synthetic sequence (i.e., a sequence that does not exist as such in nature). In the practice of the present invention, the suitable promoters include, in particular, constitutive promoters and tissue-specific promoters.

Constitutive Promoters.

In certain embodiments, a construct according to the present invention comprises a plant constitutive promoter operably linked to the nucleic acid sequence encoding ORA47. The term “constitutive promoter” refers to a promoter that is able to express nucleic acid sequences operably linked to the promoter, in every or almost every tissue of the host organism and during the entire development of this organism. Plant constitutive promoters include, but are not limited to, the Cauliflower Mosaic Virus 35S (CaMV) promoter, the CaMV constitutive promoter double 35S (pd35S), the nopaline synthase promoter, the octopine synthase promoter, the 19S promoter, the rice actin promoter and actin intron (PAR-LAR) contained in plasmid pAct-1-F4, the promoter of the rice actin 1 gene, the promoter of the gene AdH of corn, the promoter of ubiquitin of corn, and the promoter pUbil of the gene encoding ubiquitin 1 of corn. Such promoters may be obtained from genomic DNA using PCR, and may then be cloned in an expression construct according to the invention.

Tissue-Specific Promoters.

In other embodiments, the expression of ORA47 is targeted to certain tissues of the transgenic plant. The term “tissue-specific promoter” refers to a promoter that is able to express, in a selective manner, nucleic acid sequences to which it is operably linked, in specific tissues of the host organism. For example, the specific expression in tissues may be performed for a preferential expression of ORA47 in the leaves and/or the stems and/or the roots of plants rather than in the seeds or fruits of the plants (in order to reduce concerns and worries associated with human consumption of genetically modified organisms). Tissue-specific expression may also be used when the pathogen to which the plant is naturally sensitive to specifically attacks given tissues of the plant.

Thus, in certain embodiments, a construct according to the present invention comprises a plant tissue-specific promoter operably linked to the nucleic acid sequence encoding ORA47. Several tissue-specific gene regulators and tissue-specific promoters that can be used in plants are known in the art. Such genes include, but are not limited to, genes encoding zeine-type storage proteins (such as napin, cruciferin, β-conglycin and phaseolin), genes involved in the biosynthesis of fatty acids (including the ACP protein—acyl carrier protein, stearoyl ACP-desaturase, and desaturases of fatty acids (fad 2-1)), and other genes that are express during the embryonic development such as Bce4 (Kridl et al., Seed Science Res., 1991, 1: 209). Tissue-specific promoters that have been described include, but are not limited to, lectin (Vodkin, Prog. Clin. Biol. Res., 1983, 138: 87; Lindstrom et al., Der. Genet., 1990, 11: 160), alcohol dehydrogenase of corn (Dennis et al., Nucleic Acids Res., 1984, 12: 983), light-harvesting antenna of corn (Bansal et al., Proc. Natl. Acad. Sci. USA, 1992, 89: 3654), heat shock protein of corn, the pea small sub-unit of ribulose 1,5-biphosphate carboxylase, the mannopine synthase in the T1 plasmid, the nopaline synthase in the T1 plasmid, the chalcone isomerise of petunia (van Tunen et al., EMBO J., 1988, 7:125), the glycin-rich protein I of bean (Keller et al., Genes Dev., 1989, 3: 1639), the truncated CaMV 25S (Odell et al., Nature, 1985, 313: 810), the potato palatine (Wenzler et al., Plant Mol. Biol., 1989, 13: 347), the corn zein protein (Reina et al., Nucleic Acids Res., 1990, 18: 6425; Kriz et al., Mol. Gen. Genet., 1987, 207: 90; Wandelt et al., Nucleic Acids Res., 1989, 17 2354), the PEPC promoter of the phosphoenolpyruvate carboxylase gene of sorghum (Crétin et al., Gène, 1991, 99: 87-94), the HMGW promoter of wheat (Blechl and Anderson, Nat. Biotech., 1996, 14: 875-879) or barley, and the promoters of chalcone synthase (Franken et al., EMBO J., 1991, 10: 2605).

Transcription Termination Regions.

In the practice of the present invention, the transcription termination region present in the expression construct may be of the same origin as (i.e., be homologous to) the transcription initiation region or the nucleic acid sequence encoding ORA47, or of different origin (i.e., heterologous). Transcription termination regions are for example available from the Agrobacterium tumefaciens T₁ plasmid, such as the termination regions of the octopine synthase and nopaline synthase (An et al., Plant Cell, 1989, 1: 115-122; Guerineau et al., Mol. Gen. Genet., 1991, 262: 141-144; Proudfoot, Cell, 64, 671-674; Sanfacon et al., Genes Dev., 1991, 5: 141-149; Mogen et al., Plant Cell, 1990, 2: 1261-1272; Munroe et al., Gene, 1990, 91: 151-158; Ballas et al., Nucleic Acids Res., 1989, 17: 7891-7903; and Joshi et al., Nucleic Acids Res., 1987, 15: 9627-9639). Other examples of transcription termination regions include, but are not limited to, the polyA 35S of cauliflower mosaic virus (Franck et al., Cell, 1980, 21: 285-294) and the histone gene terminator (EP 0 633 317).

Other Regulatory Sequences.

Other sequences can that be present in an expression construct according to the invention are sequences that increase the genetic expression such as introns, enhancer sequences and leader sequences.

Introns that are known to increase genetic expression in plants are, for example, introns of the gene Adh1 of corn, introns of the gene bronze1 of corn (J. Callis et al., Genes Develop., 1987, 1: 1183-1200), intron DSV of tobacco yellow mosaic (Morris et al., Virology, 1992, 187: 633) and intron of actin-1 of rice (McElroy et al., Plant Cell, 1990, 2: 163-171). Suitable enhancer sequences include, but are not limited to, transcription activator of tobacco mosaic virus TEV (Carrington et al., J. Virol., 1990, 64: 1590-1597). Non-translated leader sequences that are known to increase gene expression in plants are, for example, leader sequences of tobacco mosaic virus (TMV), of maize chlorotic mottle virus (MCMV), and of alfalfa mosaic virus (AlMV) (Gallie et al., Nucl. Acids Res., 1987, 15: 8693-8711; Skuzeski et al., Plant Mol. Biol., 1990, 15: 65-79). Other suitable leader sequences include, but are not limited to, the EMCV leader (Encephalomyocarditis 5′noncoding region; Elroy-Stein et al., PNAS USA, 1989, 86: 6126-3130), the leader of human BiP-protein (Macejack et al., Nature, 1991, 353: 90-94), and the leader AMV RNA 4 from the Alfalfa mosaic virus protein (Jobling et al., Nature, 1987, 325: 622-625).

If necessary or desired, the nucleic acid sequence encoding ORA47 may be modified to include codons that are optimized for expression in a transformed plant (Campbell et al., Plant Physiol., 1990, 92: 1-11; Muray et al., Nucleic Acids Res., 1989, 17: 477-498; Wada et al., Nucl. Acids Res., 1990, 19: 2367; and U.S. Pat. Nos. 5,096,825; 5,380,831; 5,436,391; 5,625,136, 5,670,356 and 5,874,304). The sequences of such modified codons are generally synthetic sequences.

Additional Sequences.

In certain embodiments, an expression construct according to the present invention further comprises one or more marker genes. Marker genes are genes that confer a distinct phenotype to cells expressing said marker gene, which distinguishes cells that have been transformed from cells that have not been transformed. These marker genes encode a selection marker. A distinct phenotype may be used to identify plant cells, group of plant cells, plant tissues, plant organs, parts of plants or whole plants that contain in their genome an expression construct. Numerous examples of marker genes are known in the art. Some markers confer an additional advantage to the transgenic plant, such as for example resistance to a herbicide, to diseases, to bioagressors or to environmental stress.

Examples of markers that confer a resistance to herbicides and that can be used in the practice of the present invention include, but are not limited to, the gene bar of Streptomyces hygroscopicus which encodes phosphinothricin acetylase (PAT) providing a resistance to glufosinate, mutant genes that confer a resistance to imidazalinone or to sulfonylurea such as the genes encoding the mutant form of the ALS and AHAS enzymes (Lee et al., EMBO J., 1988, 7: 1241; Miki et al., Theor. Appl. Genet., 1990, 80: 449; and U.S. Pat. No. 5,773,702), genes that confer a resistance to glycophosphate such as the mutant forms of EPSP synthase and aroA, a resistance to L-phophinothricine such as the genes of glytamine synthase, a resistance to kanamycin such as the nptI and nptII genes of omycin phosphotransferase, or a resistance to phenoxypropionic acids and to cyclohexones such as the genes encoding the ACCAse inhibitor (Marshall et al., Theor. Appl. Genet., 1992, 83: 435).

Marker genes that confer a resistance to diseases or to bioagressors and which may be used in the practice of the present invention include, but are not limited to, genes encoding a protein of Bacillus thuringiensis such as delta-endotoxin (U.S. Pat. No. 6,100,456); genes encoding proteins that bind to vitamins such as avidine and homologs thereof that are used as larvicides against insects; genes encoding protease inhibitors or amylase inhibitors such as rice cystein proteinase (Abe et al., J. Biol. Chem., 1987, 262: 16793) and tobacco proteinase inhibitor I (Hubb et al., Plant Mol. Biol., 1993, 21: 985); genes encoding hormones specific of insects or of pheromones such as ecdysteroid hormone or juvenile hormone and equivalents thereof; genes encoding peptides or neuropeptides that are specific of insects and whose expression disturb said insects' physiology; genes encoding venom specific of insects, genes encoding enzymes responsible for the accumulation of monoterpenes, sesquiterpenes, hydroxamic acid, phenylpropanoid derivative or other non-proteinic molecules that exhibit an insecticidal activity, genes encoding enzymes involved in the modification of the biological activity of a molecule (U.S. Pat. No. 5,539,095); genes encoding hydrophobic peptides such as Tachyplesin derivatives that inhibit fungal pathogens; genes encoding a viral invasive protein or a toxin derivative (Beachy et al., Ann. Rev. Phytopathol., 1990, 28: 451); and genes encoding an antibody or antitoxin specific of insects or an antibody specific of a virus (Tavladoraki et al., Nature, 1993, 366: 469).

Marker genes that confer a resistance to environmental stress and that may be used in the practice of the present invention include, but are not limited to, mtld and HVAI; rd29A et rd19B, which are genes of Arabidopsis thaliana encoding hydrophilic proteins that are induced in response to dehydration, low temperatures, stress due to salinity, or exposure to abscisic acid (Yamaguchi-Shinozaki et al., Plant Cell, 1994, 6: 251-26). Other examples of such genes are described in U.S. Pat. Nos. 5,296,462 and 6,356,816.

Alternatively, a marker gene may cause, in plant cells transformed or in plants transformed, a visible response (e.g., a distinctive appearance, such as a different color or different growth compared to plant cells or plants that do not express the marker gene). These marker genes encode a reporter. It is known in the art that transcription activators of the biosynthesis of anthocyanine operably linked to a suitable promoter in an expression construct is of great utility as non-phytotoxic marker for the transformation of plant cells.

The location of a protein may be altered by modifying the nucleic acid sequence encoding the protein by addition of a region encoding a signal peptide. Methods for adding such regions have been described (Dai et al., Trans. Res., 2005, 14: 627; Keegstra et al., Physiol. Plant., 1995, 93: 157-162; Zoubenko et al., Nucleic Acids Res., 1994, 22: 3819-3824; Jones et al., Plant. Physiol., 1993, 101: 595-606; Nhakamura et al., Plant. Physiol., 1993, 101: 1-5; Hemon et al., Plant Molecular Biology, 1990, 15: 895-904; Yang et al., Proc. Natl. Acad. Sci. USA, 1990, 87: 4144-4184; Thoma et al., Plant. Physiol., 1994, 105: 35-45; or WO 88/02402).

An expression construct according to the present invention may also further comprise any other nucleic acid sequence which, following transcription, confers an additional desirable property to the transformed plant obtained. Examples of such desirable properties include, but are not limited to, the ability to grow under different climate conditions and/or in different soils; incorporation of bio-confinement characteristics such as for example sterile flowers (for males only or for both males and females); incorporation of phytoremediation characteristics, and increased biomass.

### Vectors

In certain embodiments, an expression construct of the present invention is inserted into a suitable vector. As used herein, the term “vector” refers to a circular or linear, DNA or RNA molecule that is indifferently under a single strain or double strain form. A recombinant vector according to the present invention is preferably an expression vector or more specifically an insertion vector, a transformation vector or an integration vector. A vector may be of bacterial or viral origin.

In any case, in a vector according to the present invention, the nucleic acid sequence encoding ORA47 is placed under the control of one or more sequences comprising regulatory signals that regulate the expression of the nucleic acid sequence encoding ORA47 in a given plant, as mentioned above. In some embodiments, these regulatory signals are contained in the expression construct that is inserted in the vector. In other embodiments, one or more regulatory signals are contained in the expression construct and one or more other regulatory signals are contained in the vector. In yet other embodiments, all the regulatory signals are contained in the vector.

A recombinant vector according to the present invention may preferably comprise suitable transcription initiation and termination sequences. Furthermore, a recombinant vector according to the present invention may comprise one or more origin of replication sequences that are functional in plants in which their expression is desired, as well as optionally selection marker sequence(s). Recombinant vectors according to the present invention may include one or more regulatory signals as defined above. In certain embodiments, a recombinant vector according to the present invention is an integration vector that allows the insertion of multiple functional copies of the nucleic acid sequence encoding ORA47 in the plant genome.

Preferably, a vector according to the present invention is selected among those vectors specifically suitable for the expression of sequences of interest in plant cells, such as for example the cambia 1302 vector (Hajdukiiewicz et al., Plant Mol. Biol., 1994, 25: 989-994) and the vectors commercialized by Clontech; the pBIN19 vector (Bevan et al., Nucleic Acids Res., 1984, 12: 8711-8721), the pBI 101 vector (Jefferson, Plant Mol. Biol. Report., 1987, 5: 387-405), the pBI 121 vector (Jefferson, Plant Mol. Biol. Report., 1987, 5: 387-405), and the pEGFP vector (Yang et al., Nat. Biotechnol., 1996, 14: 1246-1251, Yang et al., Nucleic Acids Res., 1996, 24: 4592-4593).

Very often, vectors used for genetic transformation exist under the form of plasmids. In such cases, the term “plasmid” refers to an autonomous circular DNA molecule that is capable of replication in a cell. If a microorganism or a recombinant cell culture is described as host of an expression plasmid, said plasmid comprises both extrachromosomic circular DNA and DNA having integrated host chromosome(s). If the plasmid is maintained in a host cell, the plasmid is either stably replicated during mitosis as an autonomous structure, or is integrated into the host's genome. Plasmids that may be used in the practice of the present invention include, but are not limited to, the Ti plasmids of Agrobacterium tumefaciens (Darnell, Lodish, Baltimore, “Molecular Cell Biology”, 2^{nd} Ed., 1990, Scientific American Books: New York), a plasmid comprising a β-glucuronidase gene and a Cauliflower mosaic virus (CaMV) promoter with a leader sequence from the Alfalfa Mosaic virus (Sanford et al., Plant Mol. Biol., 1993, 22: 751-765) and a plasmid comprising a bar gene cloned downstream of a CaMV 35S promoter with a leader sequence from the Tobacco Mosaic virus (TMV). Some plasmids may comprise introns, such as those derived from alcohol dehydrogenase (Adh1) and other DNA sequences. The size of the vector is not a limiting factor.

When the expression constructs or vectors are intended to be used in the transformation via Agrobacterium tumefaciens, the plasmid may comprise an origin of replication that allows replication in Agrobacterium and a high number of origins of replication that are functional in E. Coli. This allows for the easy production and testing of transgenes in E. Coli before transfer to Agrobacterium for subsequent introduction into plants.

### Preparation of Expression Constructs or Expression Vectors

Irrespective of the components of the expression construct or expression vector, one skilled in the art will understand that this construct or vector may be prepared using any of a variety of suitable methods, the method used for preparing the expression construct or vector being a non-critical or limiting element of the invention.

Methods for the preparation of such nucleic acid constructs are known in the art and have been described, for example in different textbooks such as Sambrook, Fritsch and Maniatis, “Molecular Cloning: A Laboratory Manual”, 1989, Cold Spring Harbor Laboratory: Cold Spring Harbor, et Silhavy, Berman, and Enquist, “Experiments with Gene Fusions”, 1984, Cold Spring Harbor Laboratory: Cold Spring Harbor; F. M. Ausubel et al., “Current Protocols in Molecular Biology”, 1989, John Wiley & Sons: New York.

### II—Methods for Preparing Plants Exhibiting an Overproduction of Jasmonic Acid

Expression constructs or expression vectors described herein may be used to obtain transgenic plants exhibiting an overproduction or accumulation of jasmonic acid. Therefore, in one aspect, the present invention provides methods of the preparation of such transgenic plants. As used herein, the term “transgenic plant” refers to a plant that has been obtained using techniques involving genetic manipulations. More specifically, a transgenic plant is a plant with (i.e. containing) at least one cell comprising heterologous nucleic acid sequences that were introduced by the hand of man. Typically, transgenic plants express DNA sequences which confer to these plants one or more characters that are different from those of non-transgenic plants of the same species.

The present invention generally provides a method for obtaining a transgenic plant that overproduces or accumulates jasmonic acid, said method comprising transforming a plant using an expression construct comprising a nucleic acid sequence encoding ORA47, or an expression vector comprising said expression construct. The terms “overproduction of jasmonic acid”, “accumulation of jasmonic acid” and related terms are used herein interchangeably. They refer to a production or accumulation of jasmonic acid in the transformed plant that is higher than the production or accumulation of jasmonic acid in a non-transformed plant of the same species and at the same development stage. In certain embodiments, the production of jasmonic acid in the transformed plant is at least 2 times higher than that in the non-transformed plant, preferably at least 5 times higher, at least 10 times higher, at least 25 times higher, at least 50 times higher, at least 75 times higher, at least 100 times higher, or more than 100 times higher than the production or accumulation of jasmonic acid in the non-transformed plant.

In certain embodiments, a transformed plant of the present invention that overproduces jasmonic acid also overproduces or accumulates OPDA, a jasmonic acid precursor. As above, the terms “overproduction of OPDA”, “accumulation of OPDA” and related terms refer to a production or accumulation of OPDA in the transformed plant that is higher than the production or accumulation of OPDA in a non-transformed plant of the same species and at the same development stage. In certain embodiments, the production of OPDA in a transformed plant is at least 2 times higher than that in the non-transformed plant, preferably at least 5 times higher, at least 10 times higher, at least 15 times higher, at least 25 times higher, at least 30 times higher, at least 40 times higher, at least 50 times higher, at least 75 times higher, at least 100 times higher or more than 100 times higher than the production or accumulation of OPDA in the non-transformed plant.

Transformation of a plant using an expression construct or expression vector may be performed using any suitable method, since the transformation method used is not critical to the present invention. Suitable methods include, but are not limited to, non-biological methods (e.g., micro-injection, microprojectile bombardment, electroporation, infiltration under vacuum, or direct precipitation) and biological methods (e.g., infection with a transformed bacterial strain such as an Agrobacterium strain). Alternatively, any combination of these methods that allows for an efficient transformation of plant cells or of plants may be used in the practice of the present invention.

### General Methods of Transformation

Thus, in certain embodiments, a method for obtaining a transgenic plant that overproduces or accumulates jasmonic acid comprises steps of: (a) transforming a plant cell with an expression construct or expression vector comprising a nucleic acid sequence encoding ORA47 in order to obtain a plant cell stably transformed; and (b) culturing the plant stably transformed in order to regenerate a whole plant comprising, integrated within its genome, a nucleic acid sequence allowing the expression of ORA47 in the plant. The culturing step may comprise regenerating several plants and selecting, among the regenerated plants, those plants that comprise, integrated within their genome, a nucleic acid allowing the expression of ORA47 in the plant.

In other embodiments, a method for obtaining a transgenic plant that overproduces or accumulates jasmonic acid comprises: (a) transforming an Agrobacterium tumefaciens or Agrobacterium rhizogenes host cell in order to obtain a recombinant host cell; and (b) transforming a plant or plant cell via infection with the recombinant host cell in order to obtain a whole plant comprising, integrated within its genome, a nucleic acid sequence allowing the expression of ORA47 in the plant. The first transformation step may comprise: infecting several plants or several plant cells with recombinant host cells; optionally culturing several infected plant cells in order to regenerate several plants; and selecting, among the infected plants or among the regenerated plants, those plants that comprise, integrated within their genome, a nucleic acid allowing the expression of ORA47 in the plant.

In certain particular embodiments, these methods may further comprise the following additional steps: (c) crossing two transformed plants in order to obtain crossed plants; and (d) selecting, among the crossed plants obtained, those plants that are homozygous for the transgene.

Alternatively, these methods may further comprise the following additional steps: (c) crossing a transformed plant with a plant of the same species in order to obtain hybrid plants; and (d) selecting, among the hybrid plants obtained, those plants that have conserved the transgene.

### Transformation of Plant Cells and Host Cells

As used herein, the term “plant cell” include protoplasts (plant cells without walls), plant germ cells or somatic cells, and more generally any cell or cell group capable of regenerating a whole plant. Thus, a seed which comprises multiple plant cells and which can regenerate a whole plant is encompassed within the term “plant cell”. A cell plant used in a method of the present invention may be isolated from the plant from which it originates (e.g., cell line) or from the culture of a plant tissue or organ. Plant cells used in a method of the present invention may originate from any plant that does not belong to the Arabidopsis thaliana species (see below).

Plant cells may be obtained from a large number of different sources such as the American Type Culture Collection (Rockland, Md.) or from other commercial sources of seeds such as for example A. Atlee Burpee Seed Co. (Warminster, Pa.), Park Seed Co. (Greenwood, S.C.), Johnny Seed Co. (Albion, Me.), or Northrup King Seeds (Hartsville, S.C.), Vilmorin, France, Thompson & Morgan, Graines Baumaux: Clause vegetable seeds.

Host cells useful for the transformation of plants are described in I. K. Vasil, “Cell Culture and Somatic Cell Genetics of Plants”, Vol. I, II and II; 1984, Laboratory Procedures and Their Applications Academic Press: New York; R. A. Dixon et al., “Plant Cell Culture—A Practical Approach”, 1985, IRL Press: Oxford University; and Green et al., “Plant Tissue and Cell Culture”, 1987, Academic Press New York.

Transformation of plant cells (or of host cells) may be performed using any method known to those skilled in the art. Methods for introducing expression constructs in plant cells have been described. See, for example, “Methods for Plant Molecular Biology”, Weissbach and Weissbach (Eds.), 1989, Academic Press, Inc; “Plant Cell, Tissue and Organ Culture: Fundamental Methods”, 1995, Springer-Verlag: Berlin, Germany; and U.S. Pat. Nos. 4,945,050; 5,036,006; 5,100,792; 5,240,855; 5,302,523; 5,322,783; 5,324,646; 5,384,253; 5,464,765; 5,538,877; 5,538,880; 5,550,318; 5,563,055; and 5,591,616). Other methods for the direct transfer of genes are also of interest such as direct microinjection of embryoid (Neuhaus et al., Theoretical and Applied Genet., 1987, 75: 30-36), infiltration under vacuum (Bechtold et al., Comptes Rendus Acad. Sci. Série III—Sciences de la Vie, 1993, 316(10), electroporation (Chupeau et al., Biotechn., 1989, 7: 503-508), direct precipitation using PEG (Schocher et al., Biotechn., 1986, 4: 1093-1096) or DNA particle bombardment (Fromm et al., Biotechn., 1990, 8: 833-839).

In particular, electroporation has often been used to transform plant cells (U.S. Pat. No. 5,384,253). This method is generally carried out on friable tissues (such as, for example, a suspension of cells or an embryogenic callus) or embryo cells or other organized tissues that have been rendered more susceptible to electroporation by exposition to enzymes that degrade pectin or via mechanical treatment. For example, intact cells of corn, wheat, tomato, soybean and tobacco have been transformed by electroporation (D'Halluin et al., Plant cell, 1992, 4: 1495-1505; Rhodes et al., Methods Mol. Biol. 1995, 55: 121-131; and U.S. Pat. No. 5,384,253). Electroporation can also be used to transform protoplasts (Bates, Methods Mol. Biol. 1999, 111: 359-366).

Particle bombardment techniques may be used to transform almost any species of monocotyledon or dicotyledon plant (U.S. Pat. Nos. 5,036,006; 5,302,523; 5,322,783 and 5,563,055, WO 95/06128; Ritala et al., Plant Mol. Biol. 1994, 24: 317-325; Hengens et al., Plant Mol. Biol. 1993, 23: 643-669; Hengens et al., Plant Mol. Biol. 1993, 22: 1101-1127; Buising et al., Mol. Gen. Genet. 1994, 243: 71-81; Singsit et al., Transgenic Res. 1997, 6: 169-176).

Transformation of plant protoplasts may be carried out using methods such as precipitation by calcium phosphate, treatment with polyethylene glycol, electroporation or any combination thereof (Potrykus et al., Mol. Gen. Genet. 1985, 199: 169-177; Fromm et al., Nature, 1986, 31: 791-793; Callis et al., Genes Dev. 1987, 1: 1183-1200; Omirulleh et al., Plant Mol. Biol. 1993, 21: 415-428).

Other methods for transforming plant cells that are known or have been described in the art (for example Rakoczy-Trojanowska, Cell Mol. Biol. Lett. 2002, 7: 849-858) may alternatively or additionally be used in the practice of the present invention.

The transformation of plant cells via Agrobacterium is also well known in the art (U.S. Pat. No. 5,563,055). This method has been used for the transformation of dicotyledons and monocotyledons. It is often the method of choice for plant species for which transformation via Agrobacterium is efficient. Transformation via Agrobacterium is performed in several steps: first, cloning and DNA modifications are carried out in E. Coli, then the plasmid comprising nucleic sequences of interest is transferred by conjugation or electroporation in a bacterial strain of Agrobacterium (generally Agrobacterium tumefaciens or Agrobacterium rhizogenes), and the recombinant Agrobacterium cells obtained are used to infect plants or plant cells. For example, plant cells that can be transformed using this method are typically callus cells, embryo cells, meristematic cells, or cell cultures in suspension.

Preferably, in a method of transformation according to the present invention, plant cells are stably transformed. The term “stably transformed”, as used herein, refers to a cell, a callus or a protoplast in which an exogenous nucleic acid molecule that has been introduced by a method of transformation is capable of replication. The stability of the transformation is demonstrated by the ability of the transformed cell to establish cell lines or clones comprising a population of daughter cells that also comprise the exogenous nucleic acid molecule.

The success of the transformation of a plant cell may preliminarily be evaluated visually when the expression construct or expression vector used comprises a marker gene (as described herein). Alternatively, plant cells which comprise the nucleic acid sequence encoding ORA47 and which express ORA47 may be identified and selected using any of a variety of suitable procedures such as via DNA-DNA or DNA-RNA hybridizations, protein or immunologic assays known to detect and quantify nucleic acids and proteins.

Plant cells (including protoplasts, calluses, etc. . . . ) stably transformed by a method of the present invention are also encompassed within the scope of the invention.

### Regeneration of Plants

It is known in the art that regeneration from individual transformed cells to obtain whole transgenic plants is possible for a large number of plants. Regeneration has been demonstrated in the case of dicotyledons as well as in the case of monocotyledons. In the practice of the present invention, plant cells stably transformed may be cultured to obtain transgenic plants using any standard method known in the art (see, for example, McCormick et al., Plant Cell Reports, 1986, 5: 81-84). Regeneration of plants from protoplasts has also been described, for example by Evans et al., “Handbook of Plant Cell Cultures”, Vol. 1, 1983, MacMilan Publishing Co: New York; and I. R. Vasil (Ed.), “Cell Culture and Somatic Cell Genetics of Plants”, Vol. I (1984) and Vol. II (1986), Acad. Press: Orlando. As used herein, the term “regeneration” refers to a process whereby a plant is grown from a plant cell.

Means of regeneration may vary from one plant species to another. However, generally, a suspension of transformed plant cells or of transformed explants contained in a Petri dish is used. A plant callus is formed, from which appear sprouts and then roots. Alternatively, a technique of somatic embryogenesis may be used. Using this technique, it is possible from a single seed or a single callus to obtain an unlimited number of copies of this seed or callus, wherein each of the copies is morphologically and genetically identical to the starting seed or callus.

Primary transgenic plants may be cultivated using conventional methods. Numerous techniques for plant cultivation are known in the art. Thus, the plants of the present invention may be cultivated in the soil, or alternatively may be grown via hydroponic cultivation (i.e., in the absence of soil—see, for example, U.S. Pat. Nos. 5,364,451; 5,393,426; and 5,785,735).

### Selection of Transformed Plants

Selection of plants that have been transformed may be carried out using any suitable method, for example Northern Blot, Southern Blot, detection of resistance to a herbicide or to an antibiotic agent, or any combination of these methods or other methods known to those skilled in the art. The techniques of Southern Blot and Northern Blot, which respectively test the presence, here in a plant tissue, of a nucleic acid sequence of interest (e.g., sequence encoding ORA47) and of the corresponding RNA, are standard methods known in the art (see, for example, Sambrook & Russell, “Molecular Cloning”, 2001, Cold Spring Harbor Laboratory Press: Cold Spring Harbor). Alternatively or additionally, the selection may be carried out based on the detection of an overproduction of jasmonic acid (see Examples).

Primary transformed (and optionally selected) transgenic plants may be crossed among themselves, or crossed with plants of the same species. Then plants that exhibit desired phenotypic characteristics may be selected among the crossed plants or hybrid plants obtained. Several generations of plants may be generated in order to ensure that the desired phenotypic characteristics are indeed inherited and stably maintained, and seeds of these plants may then be harvested.

For example, a plant transformed according to the present invention may be crossed with a plant of the same species but which is considered to be of high agronomic value. Hybrid plants obtained that have conserved the transgene may then be submitted to another crossing procedure with the plant of high agronomic value in order to obtain plants that have conserved the transgene and that possess a genetic background that is close or identical to the genetic background of the plant of high agronomic value.

### III—Transgenic Plants Exhibiting an Overproduction of Jasmonic Acid

The present invention also provides transgenic plants obtained by a method described herein, i.e. transgenic plants comprising, integrated within their genome, an exogenous nucleic acid sequence allowing the expression of ORA47 in the plant. In particular, transgenic plants of the present invention are characterized in that the expression of ORA47 induces an overexpression or accumulation of jasmonic acid. As mentioned above, an overproduction or accumulation of jasmonic acid in a transgenic plant corresponds to a production or accumulation of jasmonic acid in the transformed plant which is higher that the production or accumulation of jasmonic acid observed in a plant of the same species and at the same development stage but which has not been transformed. In certain embodiments, the overproduction of jasmonic acid in the transformed plant is at least 2 times higher, at least 5 times higher, at least 10 times higher, at least 25 times higher, at least 50 times higher, at least 75 times higher, at least 100 times higher or more than 100 times higher than the production or accumulation of jasmonic acid in the non-transformed plant.

In certain embodiments, a transgenic plant according to the present invention is also characterized in that the expression of ORA47 induces an overproduction or accumulation of OPDA, a jasmonic acid precursor. As mentioned above, an overproduction or accumulation of OPDA in a transgenic plant corresponds to a production or accumulation of OPDA in the transgenic plant that is higher than the production or accumulation of OPDA in a non-transformed plant of the same species and at the same development stage. In certain embodiments, the overproduction of OPDA in the transformed plant is at least 2 times higher, at least 5 times higher, at least 10 times higher, at least 20 times higher, at least 25 times higher, at least 30 times higher, at least 40 times higher, at least 50 times higher, at least 75 times higher, at least 100 times higher or more than 100 times higher than the production or accumulation of OPDA in the non-transformed plant.

With the exception of the Arabidopsis thaliana species, transgenic plants of the present invention may belong to any plant genus or plant species for which a transformation via introduction of an expression construct or expression vector comprising a nucleic acid sequence encoding ORA47 results in overproduction or accumulation of jasmonic acid. Thus, transgenic plants of the present invention may be plants of large cultures, vegetables, flowers or trees. Transgenic plants of the present invention may be dicotyledons, such as Malvaceae (e.g., Cotton, etc. . . . ), Solanaceae (e.g., tobacco, tomato, potato, eggplant, etc. . . . ), Cucurbitaceae (e.g., melon, cucumber, watermelon, squaches, etc. . . . ), Brassicaceae (e.g., colza, mustard, etc. . . . ), Asteraceae (e.g., cichorium, etc. . . . ), Apiaceae (e.g., carrot, cumin, etc. . . . ), Rosaceae (in particular trees and arbusts whose fruits are economically valuable) or monocotyledons, such as for example in particular cereals (e.g., wheat, barley, oat, rice, corn, etc. . . . ) or liliaceae (e.g., onion, garlic, etc. . . . ).

In certain embodiments, a transgenic plants of the present invention belongs to the Malvaceae family (e.g., cotton, cocoa, okra, etc. . . . ), to the Solanaceae family (e.g., tobacco, tomato, potato, eggplant, etc. . . . ), to the Rubiaceae family (e.g., coffee, etc. . . . ), to the Poaceae or Gramineae family (e.g., rice, corn, wheat, barley, oat, rye, mil, sugarcane, etc. . . . ) or to the Vitaceae family (e.g., vine, etc. . . . ).

In certain preferred embodiments, a transgenic plant of the present invention belongs to the Gossypium or Cotoneaster genuses (cotton), to the Nicotiona genus (tobacco), to the Oryza genus (rice), to the Solanum genus (tomato), to the Coffea genus (coffee), or to the Vitis genus (vine). In particularly preferred embodiments, a transgenic plant of the present invention belongs to the Gossypium or Cotoneaster genuses (cotton). In other particularly preferred embodiments, a transgenic plant of the present invention belongs to the Nicotiona genus (tobacco).

The invention encompasses whole transgenic plants, their progeny (or descendants) including cross-progeny, as well as any vegetal material obtained from these plants. The term “vegetal material”, as used herein, includes plant cells, plant organs, protoplasts, plant calluses, cultures of plant cells or other plant cells organized as functional and/or structural units, plant seeds, leaves, stems, roots, flowers, fruits, tubers, pollen, plant cuttings and the like.

IV—Use of Transgenic Plants that Overproduce Jasmonic Acid

### Improved Resistance to Pathogenic Agents

Given the important role of jasmonic acid in the mechanisms of plant defense, in particular against insects and bacteria but also against fungi, as mentioned above, the methods according to the present invention may be used to generate plants exhibiting an improved resistance to pathogenic agents. As used herein, the term “improved resistance to pathogenic agent” refers to the resistance of a transformed plant (i.e. a capability to defend itself) against at least one pathogenic agent which is higher than the resistance exhibited by a plant of the same species that has been transformed. In the context of the present invention, a pathogenic agent may be any one of a variety of microorganisms (bacteria, fungi, mycoplasma, viruses), insects and other bioaggressor capable of causing a disease in a plant.

### Production of Secondary Metabolites

A lot of plant secondary metabolites have an economic value as pharmaceutical products (e.g., taxol, digoxine, colchicine, codeine, morphine, quinine, quinidine, shikonine, ajmaline, ajmalicine, vinblastine, vincristine, reserpin, rescinnamine, camptothecine, ellipticine, nicotine, etc. . . . ), colorants or food flavors (e.g., anthocyanins, vanillin, etc. . . . ), and fragrances. However, the industrial use of these secondary metabolites is limited by the fact the plants only produce low quantities of these metabolites.

The biosynthesis of numerous classes of secondary metabolites is stimulated by jasmonates and some of their precursors (Memelink, Curr. Opin. Plant Biol., 2005, 8: 23-235). In particular, in certain plants, jasmonic acid induces the expression of genes involved in the biosynthesis of secondary metabolites (Menke et al., EMBO J., 1999, 18: 4455-4463). A non-exhaustive list of classes of secondary metabolites whose biosynthesis is induced by jasmonates includes taxoids, phenylpropanoids, flavanoids, anthocyanins, guaianolides, anthraquinones, sesquiterpenoids, and several types of alkaloids such as terpenoid indole alkaloids.

Therefore, the transgenic plants according to the present invention, which overproduce jasmonic acid, can be used for the production of secondary metabolites. Similarly, the methods according to the present invention can be used to produce plants that overproduce at least one secondary metabolite whose biosynthesis is induced by jasmonates.

Any plant known to produce a secondary metabolite whose synthesis is induced by jasmonates, and in particular by jasmonic acid, can be transformed using a method of the present invention with the goal of obtaining a transgenic plant which overproduces said secondary metabolite. Examples of plants known to produce such secondary metabolites include, but are not limited to, Madagascar Periwinkle (Catharanthus roseus) whose leaves synthesize vinblastine and vincristine—compounds that are used in the treatment of cancer—and whose roots synthesize ajmalicine, which is used to improve cerebral blood flow; opium poppy (Papaver somniferum), which produces latex comprising narcotic alkaloids such as morphine and codeine; Rauwolfia serpentine, which produces several bioactive compounds such as reserpine which is used as a hypotensive agent; plants from the Cinchona genus which produce quinine used in the treatment of malaria and quinidine, an antiarrythmic agent; henbanes such as the White Henbane (Hyoscyamus albus L.) and the Black Henbane (Hyoscyamus niger L.) or plants of the Datura genus which comprise several alkaloids such as atropine, a cholinergic antagonist used for example to reduce the shakings in patients suffering from Parkinson's disease, hyoscyamine which is used in the treatment of gastrointestinal diseases, and scopolamine, which is a central sedative; tobacco which produces nicotine, which is known to have useful therapeutic effects in neurologic and psychiatric conditions.

Unless otherwise defined, all the technical and scientific terms used herein have the same meaning as generally understood in the field. All the publications, patent applications, patents and other references mentioned herein are each incorporated herein by reference in its entirety.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data are actually obtained.

### Example 1

### Transformation of a Cotton Plant

The Agrobacterium tumefaciens strain LBA1119 has been used to transitorily transform a cotton plant. The transformation system used is called “ternary”. For numerous plant species, this system causes an increase in the frequency of T-DNA transfer (Van der fits et al., Plant Mol. Biol., 2000, 43: 495-502). Briefly, after contact between the agrobacterium and wounded plant cells, phenolic compounds, oses and an acid pH created an environment that favored the induction of the genes vir. The transcription factor VirG activated by phosphorylation can induce the expression of the vir genes which is necessary to the transfer of T-DNA. The ternary system used a mutated form of the virG protein (virGN54D) which mimics the active form.

This system was introduced in the agrobacterium strain LBA1119. This strain also possesses one of the four following binary vectors: pCAMBIA1300-GUS-intron and pCAMBIA1300-ORA47, pMDC32-ORA47 and pMDC32-GFP. The strains were spread and cultured for 48 hours at 29° C. in LB medium comprising three antibiotics: Gentamicin (Gen), Rifampicin (Rif) and Kanamycin (Kan). Some colonies were taken and cultured in liquid (medium comprising 20 mL of LB, 20 μL of Gen, 20 μL of Rif and 20 μL of Kan). Then, the culture was agitated at 200 rpm for 18 hours at 29° C. The level of agrobacteria was then measured via spectrophotometry at 600 nm. When the optical density (OD) measured was found to be between 0.6 and 1 (without dilution), then a centrifugation was performed at 3000 g at 4° C. for 20 minutes. An infiltration medium (comprising MgSO₄ (10 mM), Acetosyringone (200 μM) and MES pH 5.5 (20 mM)) was added to the pellet obtained by centrifugation in order to get an OD equal to 0.5.

Agroinfiltration was carried out on 10 days old cotyledons (cotton plants). Each cotyledon was inoculated using a needle-less syringe on the lower face. Infiltration was performed from bottom to top and between 2 main veins in order for the inoculum to spread over the whole surface of the demi-cotyledon. For each construction, 6 demi-cotyledons were agroinfiltrated. Two days (48 hours) later, the demi-cotyledons were collected and rapidly placed in liquid nitrogen. Storage was at −80° C. The experiment was repeated three times. No visible effects were observed on the cotyledons that were inoculated for 48 hours following transformation with the two constructions.

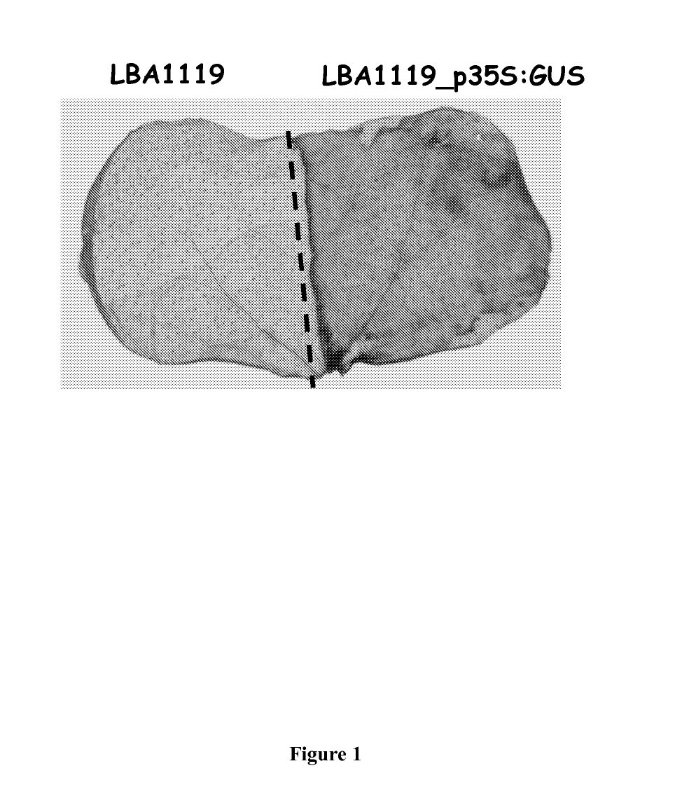

Histochemical analyses showed the presence of indigo crystals over the whole region that had been infiltrated using the LBA1119 strain containing the 13-glucuronidase reporter gene including an intron and placed under the control of a strong promoter (35S) (see FIG. 1). A qualitative and quantitative analysis (not shown) of the activity of β-glucuronidase showed that the agroinfiltration combined with the ternary system was an efficient approach for the genetic transformation of cotton plants.

### Example 2

### ORA47 Overexpression Increases Endogenous Levels of OPDA and of Jasmonic Acid in Cotton Plants

OPDA and Jasmonic Acid Extraction.

First, the vegetal material obtained from a transformed cotton plant was finely ground using a mortar in liquid nitrogen, and 250 mg of the powder obtained were placed in a 1.5 mL-Eppendorf tube. Methanol (1 mL) was added to the powder as were 100 ng of deuterated jasmonic acid (d6Ja) and 100 ng of deuterated OPDA (d50PDA), which served as internal controls. The methanolic extract was then centrifuged at 5000 g for 10 minutes at 4° C. and the supernatant was recovered in glass tubes. This extraction step was repeated three times and the methanolic extracts were combined. The methanol was then eliminated by evaporation under nitrogen (40° C. for 1 to 1.5 hours). The dried residues obtained were taken up with 5 mL of phosphate buffer (phosphate sodium 100 mM, pH 7.8, NaCl 5%). An extraction was then performed on the aqueous extract using 2.5 mL of hexane, and the hexane-phase (upper phase) was then eliminated. This extraction was repeated three times. The purified aqueous phase was then acidified at pH 1.4 using a solution of HCl 5N.

A second purification by extraction was performed using 2.5 mL of chloroform and the chloroform-phase (lower phase) was recovered using a Pasteur pipette in a different series of glass tubes. This purification step by extraction was repeated three times and the chloroform phases were combined. Then, the chloroform was eliminated by evaporation under nitrogen (40° C. for about 10 to 30 minutes) and the tubes were stored at −80° C.

Dosage of Jasmonic Acid and OPDA Extracted by LC-MS.

Analyses were performed using a Waters 1515 liquid chromatography (LC) coupled to a Waters ZQ mass spectrometer (MS) controlled by the software MassLynk V5.0 5 (MicroMass, Cray, USA). The column used was a XTerra MS C18, 3.5 μm, 100×2.1 mm (Waters, Milford, Mass., USA).

The vegetal extract was taken up with 100 μL of methanol, and 20 μL of the resulting solution were injected in the column. The separation was performed using a gradient of formic acid 15 mM: methanol (0-2 min, 40/60% (v/v); 2-14 min, de 40/60% à 60/40%; with a flow of 0.25 μL/min). Under these conditions, the retention time of jasmonic acid and of its deuterated form was 9.3 minutes, while the retention of OPDA and of its deuterated form was 16.50 minutes. Jasmonic acid and OPDA were detected and quantified in negative ESI (Electron Spray Ionisation) mode. The analysis conditions for mass spectrometry were as follows: vaporization temperature of 120° C., source temperature of 450° C., Capillary voltage 2.5 KV, Cone voltage 20 V. The quantitation was carried out using the mode “selected ion monitoring” with ion 209 for jasmonic acid, ion 215 for deuterated jasmonic acid, ion 291 for OPDA, and ion 295 for deuterated OPDA, and using ratio of peak surfaces for jasmonic acid/deuterated jasmonic acid and OPDA/deuterated OPDA, wherein the peak surface for deuterated jasmonic acid represented 100 ng.

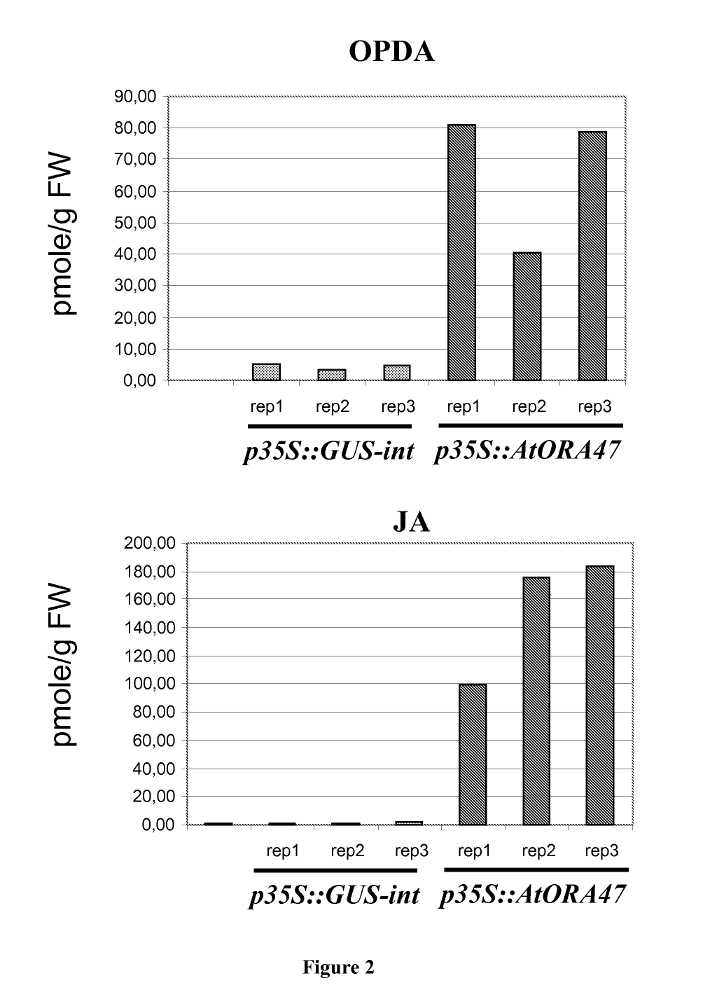

The dosage results obtained (see FIG. 2) showed a high accumulation of OPDA (40 to 80 pmole/g FW) and of jasmonic acid (100 to 180 pmole/g FW) in each of the three biological samples of cotyledons (cotton plants) that had been transformed with ORA47 compared to the samples of cotyledons (cotton plants) that has been transformed with the β-glucuronidase gene.

### Example 3

### ORA47 Overproduction in Cotton Plants Increases the Expression of Genes Involved in the Biosynthesis of Jasmonic Acid

tRNA Extraction.

Inverse transcription and quantitative real-time PCR (qPCR) analyses were performed using the samples that were prepared for the dosage of OPDA and jasmonic acid (see Example 2).

The qPCR method used was based on the detection and quantitation of a fluorescent reporter whose emission was directly proportional to the quantity of amplicons generated during the reaction. The qPCR reactions were controlled using the thermocycler MX 3500P (Stratagene, US). The detection system used SYBR Green, which binds to double stranded DNA. The quantitation of transcripts was performed using the Ct (THRESHOLD CYCLE) value, which is defined as being the threshold cycle and which is always present during the exponential phase. The more matrices are to be amplified, the higher is the Ct.

The primers used were designed using the software Beacon Designer (Premier Biosoft International, United States); their efficacy and optimal concentrations were checked. The qPCR reaction was performed using the Mesa Green qPCR Master Mix Plus for SYBER Assay (Eurogentec, Belgium) with a total volume of 20 μL (4 μL of cDNA diluted to the 10^{th}, 0.6 μL of each qPCR sense and antisense primers at 10 μM, 10 μL of Master Mix (Taq polymerase, nucleotides and Syber Green) and 4.8 μL of sterile water). The program used during the qPCR reaction was as follows: a cycle at 50° C./2 min followed by a cycle at 95° C./10 min (denaturation phase), then 40 cycles at 95° C./15 s, 58° C./20 s, 72° C./40 s and a dissociation cycle at 95° C./1 min, 60° C./30 s, 95° C./30 s. When the qPCR reaction was over, the quantitation of transcripts was performed using the software MXPro, and the values obtained were normalized to the calibrator (T0, time before infection) and to the normalizator (actin, GhACT2, Champion et al., Mol. Plant Pathol., 2009, 10: 471-485).

The expression of three genes encoding enzymes of the jasmonic acid biosynthesis pathway has been analyzed. These genes are GhAOS, GhAOC2 and GhACX1a, which encode Allene Oxyde Synthase, Allene Oxyde Cyclase 2 and Acyl-CoA Oxydase, respectively.

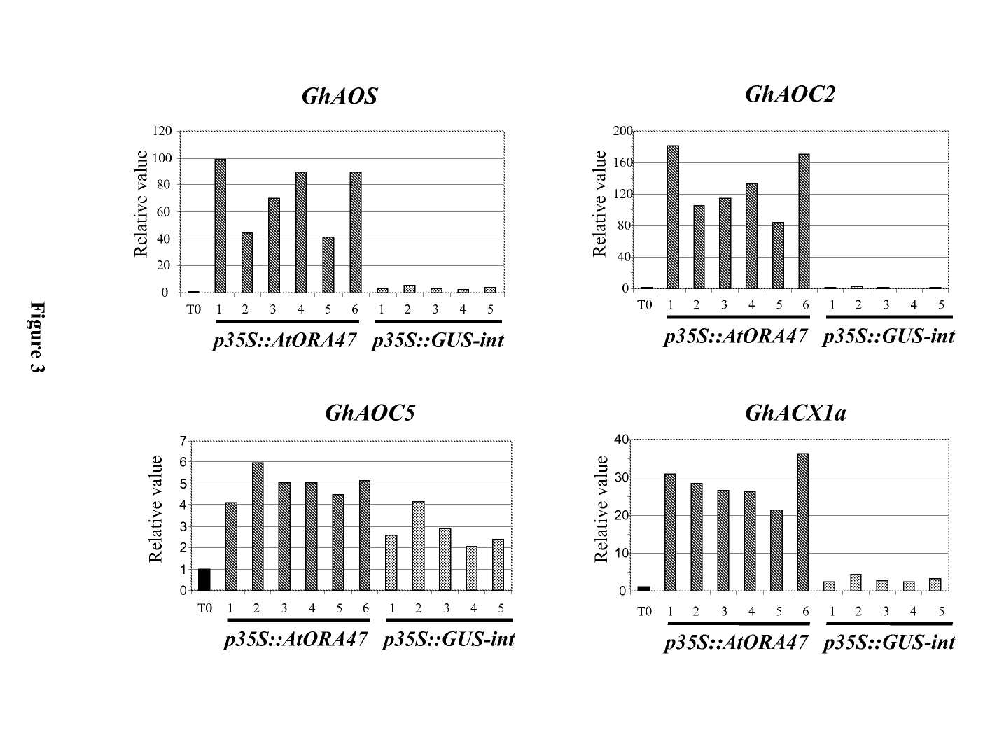

The results obtained in qPCR showed that the expression of the GhAOS gene was highly induced in plants that overexpress ORA47, with an induction maximum of 100 (see FIG. 3). Similar results were obtained for the GhAOC2 and GhACX1a genes, i.e., a high induction of the expression in plants overexpressing ORA47 (factor of 180 for GhAOC2 and factor of 36 for GhACX1a). On the other hand, in plants overexpressing the β-glucuronidase gene, no or only slight changes in the expression of these genes were observed.

### Example 4

### ORA47 Overproduction in Cotton Plants Increases the Expression of JA-responsive Genes

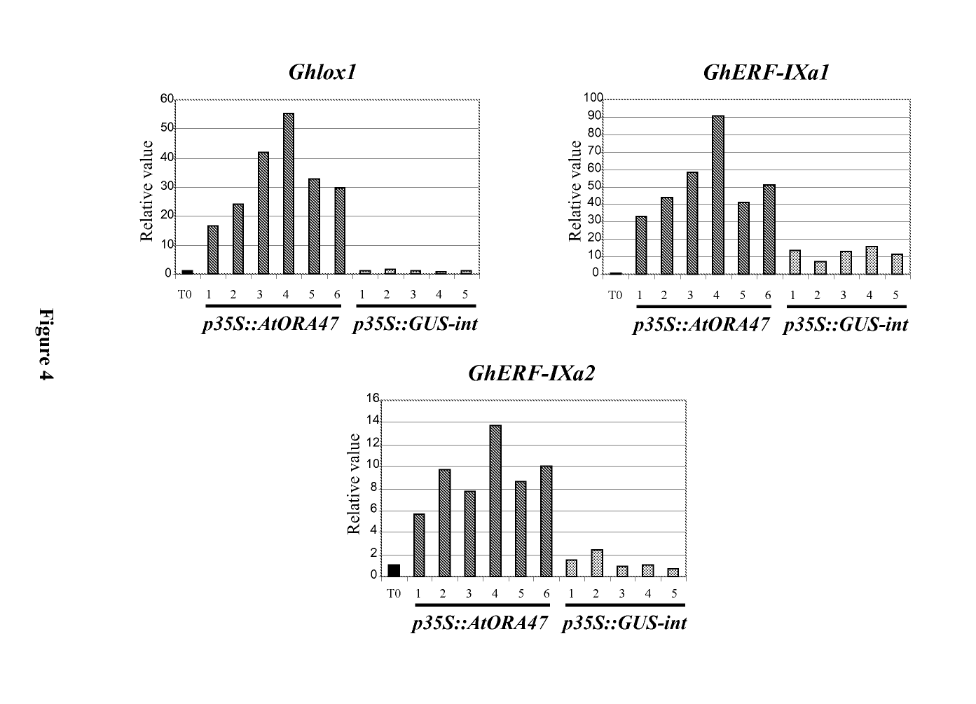

Similarly as above, the expression of 3 genes that are associated to the Cotton plant defense (Champion et al., Mol. Plant Pathol., 2009, 10: 471-485) and that are inducible by exogenous treatments of jasmonate has been analyzed. The analysis of the expression of these three genes showed an induction of the expression in response to ORA47 overexpression compared to instant T0. Little or no induction was observed in the GUS controls with the exception of GhERF-IXa1 (See FIG. 4).

### Example 5

### ORA47 is involved in Cotton Resistance against Xanthomonas campestris

Bacterial Stains.

All the different Agrobacterium tumefaciens transformed strains were maintained at 28° C. on LB agar (3.5% v/w LB+agar extract) in distilled water in the presence of antibiotics (rifampycin, 25 mg/L; kanamycin, 50 mg/L; and gentamycin, 50 mg/L). Bacteria for agro-infiltration were grown in 20 mL of LB medium (2.5% LB Broth Hight salt in distilled water) in the presence of antibiotics (rifampycin, 25 mg/L; kanamycin, 50 mg/L; and gentamycin, 50 mg/L) in a shaking incubator at 180 rpm/min at 29° C. After about 18 hours of growth, the absorbance of the different cultures was measured at 600 nm. The cultures were centrifuged for 20 minutes at 4000 min⁻¹ at 4° C. Then, the bacterial pellets obtained were resuspended in an infiltration solution (10 mM MES, 20 mM MgSO₄, pH 5.5, and 0.1 M acetosyringone) and adjusted to 10⁸ cfu/mL.

Xanthomonas campestris pv. malvacearum (Xcm) race 20 was maintained at 28° C. on LPG agar (0.5% w/v yeast extract, 0.5% w/v bacteriological peptone, 0.5% w/v glucose as a carbon source, solidified with 1.5% w/v agar; Difco, Detroit, Mich.) in distilled water. Bacteria for inoculation were grown in 20 mL LPG medium in a shaking incubator at 150 g/min, at 29° C. After about 18 hours of growth, the culture was centrifuged for 20 minutes at 4000 g/min at 4° C. and washed twice with tap water by centrifugation at 4000 g/min at 4° C. to remove nutrients and exopolysaccharide. Then, the bacteria pellet obtained was resuspended in tap water and adjusted to 10⁸ cfu/mL (the absorbance was adjusted to be 0.2 at 600 nm).

Plants Materials, Growth Conditions and Infiltrations.

Young plants of Gossypium hirsutum cv. Reba B50 containing the B2B3, two resistance genes to all races of Xcm, with the exception of race 20 were used. The plants (cotton) were grown in a greenhouse with a natural light/dark cycle at 29° C./24° C. and a relative humidity averaging 80%—conditions that are optimal for a cotton/Xcm interaction. Then, 10-day old half cotton cotyledons were agro-infiltrated with agro-bacteria using a needle-less syringe, and two days later the same half part of the cotyledons were infected with an Xcm suspension. Ten compatible Reba B-50 were transformed with GFP/Xcm (control) and ten Reba B-50 were transformed with ORA47/Xcm.

Extraction and Quantification of Xcm.

Isolations were performed using inoculated cotyledons tissues at 1, 6 and 12 days post Xcm race 20 inoculation. Densities of bacterial populations were calculated from serial dilution plate counts. Three separated sets of serial dilutions were made from each half cotyledon. Three discs (12 mm diameter) of inoculated tissues were cut from the cotyledon with a brass cork borer; the discs obtained were vertically located in three inoculated areas of a cotyledon. Discs were disinfected with 70% ethanol during 2 minutes and rinsed in two baths of sterile water. They were grounded up in 5 mL of filtered water with an Ultraturax. The suspension was serially diluted in filtered water, and triplicates were prepared for each sample. 100 μL of each of the three dilutions were plated on 3 LPG agar plates and incubated at 28° C. until colony counting.

Results.

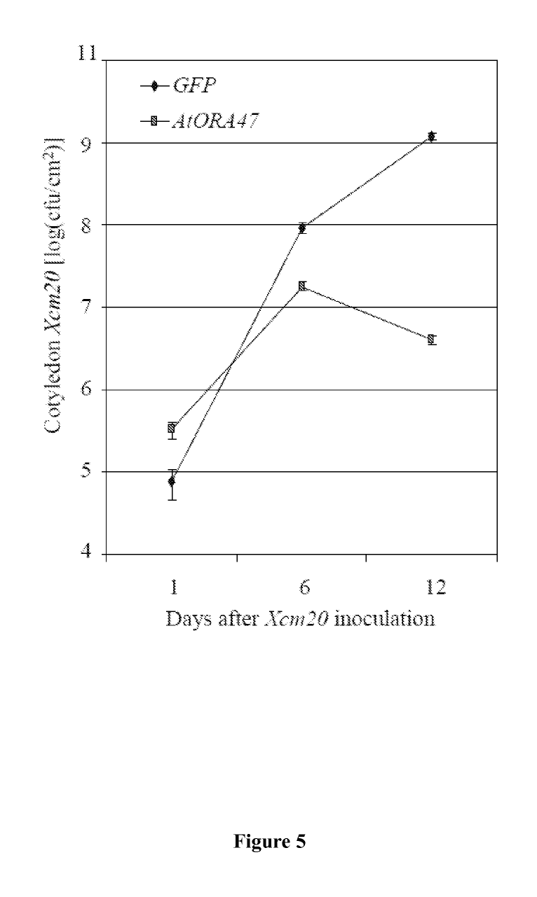

The results obtained are presented in FIG. 5, which shows the multiplication of Xcm20 in ten representative cotyledons 1, 6 and 12 days post inoculation. In GFP-transformed cotyledons, the Xcm20 population was observed to increase during the 12 days and to reach a concentration of 10⁹ cfu/cm² at day 12. In contrast, in ORA47-transformed cotyledons, the Xcm20 population was observed to increase until day 6 and then to decrease. At day 12, the Xcm20 population in ORA47-transformed cotyledons was more than 100 times lower than the Xcm20 population in the GFP-transformed cotyledons.

These results demonstrate that ORA47 plays an important role in cotton resistance to xanthomonas race 20, and that overexpression of ORA47, which leads to overproduction of ORA47 and consequently overproduction of jasmonic acid, increases resistance to these pathogenic bacteria.

### Example 6

### Accumulation of Jasmonic Acid and OPDA in Tobacco in Response to ORA47 Overexpression

Bacterial Stains.

See Example 5 above.

Plants, Plant materials, Growth Conditions and Infiltrations.

The tobacco plants (Nicotiana benthamiana) were cultivated in pots filled with nitrogen-containing potting soil in green houses under climate conditions of 24° C. during the day, 22° C. during the night, 50% relative humidity and a long-day lighting (16 h/24 h). Tobacco plants were 25 days old when they were genetically engineered. The process used to transform the tobacco leaves by agro-infiltration was the same as that used for the cotton cotyledons. The only difference was that the concentration of agro-bacterium used for the transformation of tobacco leaves was 2.5 times lower than in the case of the transformation of cotton plants.

Extraction and Dosage of Jasmonic Acid and OPDA Extracts by LC-MS.

See Example 2 above.

Results.

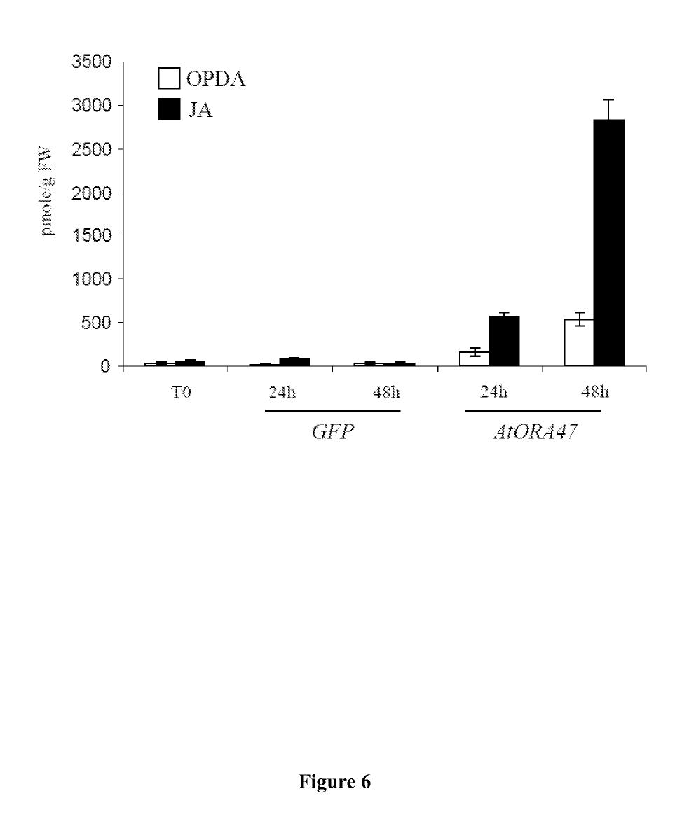

The results obtained are presented in FIG. 6. The overexpression of ORA47 in tobacco was found to induce a strong accumulation of jasmonic acid and of OPDA in the leaves transformed. The maximum accumulation of OPDA (about 500 pmole/gFW) was reached 48 hours after transformation with ORA47. Similarly, the quantity of jasmonic acid (about 3000 pmole/gFW) was found to be the highest two days after transformation with ORA47. In contrast, little or no increase in the quantity of jasmonic acid and of OPDA was detected in plant tissues transformed with GFP (control).

These results demonstrate that the control of jasmonic acid and OPDA synthesis is conserved in cotton and tobacco. The data also underline the important role played by ORA47 in the regulation of the de novo synthesis of jasmonic acid and OPDA in plants.

### Example 7

### ORA47 Overexpression in Tobacco Activates Expression of Genes Related to Nicotine Biosynthesis Enzymes

tRNA Extraction.

The method used was similar to that described in Example 3 above except that the genes tested Nubiquitine, NbAOX and NbODC were amplified using the primers described by Todd et al., Plant J., 2010, 62: 589-600. The expression data were normalized to the expression of the gene Nubiquitine.

Results.

It is known from the literature that the synthesis of nicotine, an alkaloid, is induced in response to a biotic stress in tobacco. Treatments performed with jasmonic acid have been shown to induce the expression of several genes associated with the synthesis of nicotine (Xu et al., Mol. Biol., 2004, 55: 743-761). In order to determine whether the overexpression of ORA47, which has been shown above to be responsible for the accumulation of jasmonic acid and OPDA in tobacco, also induces the expression of genes associated with the synthesis of nicotine, the expression of two genes that encode enzymes of the biosynthesis pathway of nicotine was analyzed. These two genes are NbAOX which encodes the enzyme aspartate oxydase and NbODC which encodes the enzyme ornithine decarboxylase.

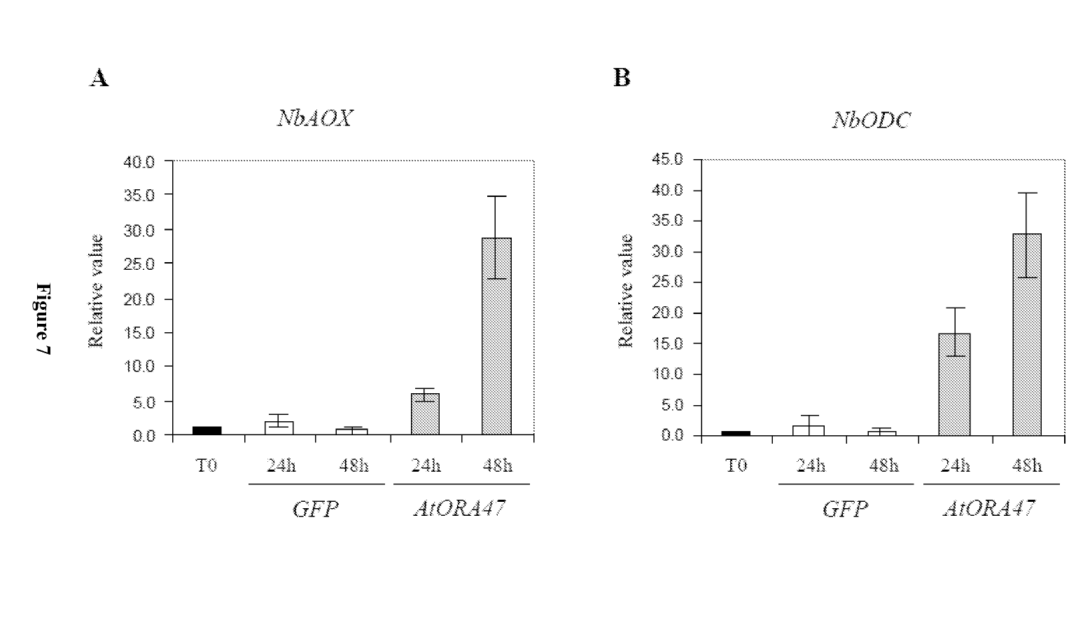

The results obtained by qPCR are presented on FIG. 7. They show an induction of the expression of the two genes NbAOX and NbODC in tobacco plants overexpressing ORA47 24 and 48 hours post agro-infiltration. In contrast, in tobacco plants transformed with GFP, little or no expression of these genes was observed.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. Transgenic plant comprising an exogenous nucleic acid allowing the expression of ORA47, wherein the expression of ORA47 induces, within the plant, an accumulation of jasmonic acid, and wherein said plant does not belong to the Arabidopsis thaliana species.

2. The transgenic plant according to claim 1, wherein expression of ORA47 also induces, within the plant, an accumulation of OPDA.

3. The transgenic plant according to claim 1, wherein the nucleic acid sequence is integrated into the genome of the transgenic plant.

4. The transgenic plant according to claim 1, wherein said plant exhibits an improved resistance to at least one pathogenic agent, wherein the pathogenic agent is selected from the group consisting of bacteria, viruses, fungi, insects and oömycetes, and any combination thereof, and wherein the pathogenic agent is capable of causing a disease in a plant.

5. The transgenic plant according to claim 1, wherein said plant belongs to the Malvaceae family, to the Solanaceae family, to the Rubiaceae family, to the Poaceae or Gramineae family or to the Vitaceae family.

6. The transgenic plant according to claim 1, wherein said plant belongs to the Gossypium genus (cotton), to the Nicotiona genus (tobacco), to the Oryza genus (rice), to the Solanum genus (tomato), to the Coffea genus (coffee), or to the Vitis genus (vine).

7. The transgenic plant according to claim 6, wherein said plant belongs to the Gossypium genus (cotton) or to the Nicotiana genus (tobacco).

8. (canceled)

9. A vegetal material obtained or extracted from a transgenic plant according to claim 1, wherein said vegetal material belongs to the group consisting of plant cells, plant cell cultures, protoplasts, plant organs, plant calluses, plant seeds, leaves, stems, roots, flowers, fruits, tubers, pollen, and plant cuttings.

10. A vegetal material according to claim 9, wherein said vegetal material is capable of regenerating a whole plant.

11-19. (canceled)

20. A method for increasing the production or accumulation of jasmonic acid in a plant that does not belong to the Arabidopsis thaliana species, said method comprising transforming the plant with an expression construct or vector comprising a nucleic acid sequence encoding ORA47 to obtain a transgenic plant.

21. The method according to claim 20, wherein the plant belongs to the Malvaceae family, to the Solanaceae family, to the Rubiaceae family, to the Poaceae or Gramineae family or to the Vitaceae family.

22. The method according to claim 20, wherein the plant belongs to the Gossypium genus, to the Nicotiana genus, to the Oryza genus, to the Solanum genus, to the Coffea genus, or to the Vitis genus.

23. The method according to claim 20, wherein the transgenic plant obtained exhibits an improved resistance to at least one pathogenic agent, wherein the pathogenic agent is selected from the group consisting of bacteria, viruses, fungi, insects, oömycetes and combination thereof, and wherein the pathogenic agent is capable of causing a disease in a plant.

24. The method according to claim 20, wherein the transgenic plant obtained further exhibits an overproduction or accumulation of OPDA.

25. The method according to claim 20, wherein transforming the plant comprises steps of:
(a) transforming a plant cell with an expression construct or expression vector comprising a nucleic acid sequence encoding ORA47 in order to obtain a plant cell stably transformed; and
(b) culturing the plant cell stably transformed in order to regenerate a whole plant comprising, integrated within its genome, a nucleic acid sequence allowing the expression of ORA47.

26. The method according to claim 25, wherein the culturing step comprises: culturing several plant cells stably transformed in order to regenerate several plants, and selecting, among the regenerated plants, those plants that comprise, integrated within their genome, a nucleic acid allowing the expression of ORA47.

27. The method according to claim 20, wherein transforming the plant comprises steps of:
(a) transforming an Agrobacterium host cell in order to obtain a recombinant host cell; and
(b) transforming a plant or a plant cell via infection with the recombinant host cell in order to obtain a whole plant comprising, integrated within its genome, a nucleic acid sequence allowing the expression or of ORA47.

28. The method according to claim 27, wherein step (b) comprises: infecting several plants or several plant cells with recombinant host cells; optionally culturing several infected plant cells in order to regenerate several plants; and selecting, among the infected plants or among the regenerated plants, those plants that comprise, integrated within their genome, a nucleic acid allowing the expression of ORA47.

29. A method for producing, in a plant, a secondary metabolite whose biosynthesis is induced by jasmonates, said method comprising transforming the plant with an expression construct or vector comprising a nucleic acid sequence encoding ORA47 to obtain a transgenic plant.

30. The method according to claim 29, wherein the plant belongs to the Nicotiana genus and the secondary metabolite is nicotine.


SEQUENCE LISTING (2 sequences)

SEQ ID NO: 1 - DNA, 918 bp, Arabidopsis thaliana
aaccagatataacagaattaacctacacaactgaaactcgagttaagaaccggaaacagaatccaattcaaccaaaccgaatcgaaccgaaccggagtttttatccaatggtgaagcaagcgatgaaggaagaggagaagaagagaaacacggcgatgcagtcaaagtacaaaggagtgaggaagaggaaatggggaaaatgggtatcggagatcagacttccacacagcagagaacgaatttggttaggctcttacgacactcccgagaaggcggcgcgtgctttcgacgccgctcaattttgtctccgcggcggcgatgctaatttcaatttccctaataatccaccgtcgatctccgtagaaaagtcgttgacgcctccggagattcaggaagctgctgctagattcgctaacacattccaagacattgtcaagggagaagaagaatcgggtttagtacccggatccgagatccgaccagagtctccttctacatctgcatctgttgctacatcgacggtggattatgatttttcgtttttggatttgcttccgatgaatttcgggtttgattccttctccgacgacttctctggcttctccggtggtgatcgatttacagagattttacccatcgaagattacggaggagagagtttattagatgaatctttgattctttgggatttttgaattcccaaacataatatttttttagagcgaactgtgagattttccttggagtcatggagaaatctggagattttttgtaacacggagctccaatgacccgggaatttctttcgtttcggatccgaatttgatgtggatcatattcacacctatattttttcatttttttgttgtaaagaaaaatcggataagattctagtaataaatgttaaaagtccatttc

SEQ ID NO: 2 - protein, 195 aa, Arabidopsis thaliana
MVKQAMKEEEKKRNTAMQSKYKGVRKRKWGKWVSEIRLPHSRERIWLGSYDTPEKAARAFDAAQFCLRGGDANFNFPNNPPSISVEKSLTPPEIQEAAARFANTFQDIVKGEEESGLVPGSEIRPESPSTSASVATSTVDYDFSFLDLLPMNFGFDSFSDDFSGFSGGDRFTEILPIEDYGGESLLDESLILWDF